(19) United States
(12) Patent Application Publication
(10) Pub. No.: US 2026/0068023 A1
(43) Pub. Date: Mar. 5, 2026

(54) PLASMA GENERATION DEVICE

(71) Applicant: FEAGLE CO., LTD, Yangsan-si (KR)

(72) Inventors: HYUNYOUNG LEE, Yangsan-si (KR); JEASHIK KIM, Seoul (KR); SONGWOONG KIM, Busan (KR); JEONGHAE CHOI, Busan (KR)

(73) Assignee: FEAGLE CO., LTD, Yangsan-si (KR)

(21) Appl. No.: 19/385,177

(22) Filed: Nov. 11, 2025

Related U.S. Application Data
(63) Continuation-in-part of application No. 18/270,838, now Pat. No. 12,494,353.

(30) Foreign Application Priority Data
Jan. 5, 2021 (KR) 10-2021-0001110
Aug. 21, 2025 (KR) 10-2025-0116246

(51) Int. Cl. H05H 1/24; A61N 1/44; B01D 53/04; B01D 53/86

(52) U.S. Cl. CPC H05H 1/24; B01D 53/0446; B01D 53/8675; A61N 1/44; B01D 2253/102; B01D 2255/2073; B01D 2257/106; B01D 2258/02; B01D 2259/4533; B01D 2259/4541; H05H 2245/17; H05H 2245/34

(57) ABSTRACT

The present disclosure discloses a plasma generation device. The device of the present disclosure includes a plasma generation pad that generates plasma and is configured such that one side based on the plasma generation pad is used as a plasma movement passage, and the other side is used as an ozone intake passage. In addition, plasma generated by the plasma generation pad is supplied to the skin through the discharge port by vertically changing a direction in the plasma movement passage, and ozone gas generated by the plasma is u-turned at the outside to be inhaled into the ozone intake passage. Ozone gas inhaled into the ozone intake passage is removed while passing through the filter, and only ozone-removed gas is discharged through the purification exhaust port.

### CROSS-REFERENCE TO RELATED PATENT APPLICATION

This application is a continuation-in-part of U.S. patent application Ser. No. 18/270,838, filed on Jul. 4, 2023, currently pending, which is a National Phase Application of PCT International Application No. PCT/KR2021/020248, which is filed on Dec. 30, 2021 and claims priority from Korean Patent Application No. 10-2021-0001110 filed on Jan. 5, 2021, which is hereby incorporated by reference as if fully set forth herein. This application also claims priority from Korean Patent Application No. 10-2025-0116246 filed on Aug. 21, 2025, which is hereby incorporated by reference as if fully set forth herein.

### TECHNICAL FIELD

The present disclosure relates to a plasma generation device, and in particular, to a plasma generation device capable of effectively removing ozone generated when air plasma is generated.

### BACKGROUND

In this specification, a plasma generation device is described using a skin beauty device as an example. But it is not limited thereto and the plasma generation device may be applied in various way to disinfection of medical equipment, oral treatment, tooth whitening, tartar removal, implant fixture surface regeneration, scalp treatment, and skin treatment.

The plasma generation device generates plasma by exciting gas for plasma generation. Conventionally, a plasma generation device required an electrode to which power is applied and a gas supply pipe to supply gas for plasma generation toward the electrode. For this reason, conventional plasma generation devices using gas were not suitable for use in beauty devices for skin care or medical devices for medical treatment.

Accordingly, Korean Patent Publication No. 10-2572882 prior art has been disclosed. A device according to the prior art is a plasma generation device that generates plasma using air. The plasma generation device that generates plasma using air in the atmosphere does not need to supply separate gas for plasma generation, and does not need to include a gas supply pipe for gas supply. Therefore, the plasma generation device is made smaller and lighter, and accordingly, it may be used suitably for a portable personal skin care device.

The plasma generation device used for skin care requires that the air plasma is in sufficient contact with the skin to enhance skin care effects, and it is also important to prevent the skin from being damaged by the plasma. That is, the plasma generation device may generate ozone gas as a byproduct during the plasma generation process, and since such ozone gas has a harmful effect on the human body, it is necessary to efficiently remove the ozone gas while using the plasma generation device. In other words, air plasma should be in direct contact with the skin surface, while ozone gas should be removed as much as possible to prevent it from reaching the skin.

### SUMMARY

### Technical Problem

The present disclosure is to solve the above-described problems, and is directed to providing a plasma generation device having a structure in which ozone gas generated as a byproduct during use of the plasma generation device may be efficiently removed.

The present disclosure is also directed to providing a plasma generation device capable of removing ozone gas as much as possible by dividing a plasma discharge port and an ozone gas intake port.

The technical problems of the present disclosure are not limited to the above-mentioned technical problems, and other technical problems not mentioned will be clearly understood by those skilled in the art from the description below.

### Technical Solution

A plasma generation device according to one embodiment of the present disclosure for solving the technical problem includes a handle and a body, wherein the body includes a plasma tip formed at a front end of the body and a plasma generation pad located inside the plasma tip and generating plasma toward an inner surface of the plasma tip, one side based on the plasma generation pad is used as a plasma movement passage for moving the plasma so that the generated plasma is transmitted to the outside through a discharge port, and the other side is used as an ozone intake passage through which ozone gas additionally generated by the plasma is u-turned at the outside to be inhaled through an intake port.

In one embodiment, the plasma generated by the plasma generation pad is discharged to the outside through the discharge port by vertically changing a direction in the plasma movement passage.

In one embodiment, the body further includes an air inflow passage through which external air flows in, and the air inflow passage may be connected to the plasma movement passage.

In one embodiment, the body further includes a purification exhaust port formed at a rear end of the body and a filter and a driving fan sequentially disposed between a plate and the purification exhaust port, and ozone is removed from the ozone gas inhaled into the ozone intake passage by a rotational force of the driving fan through the filter and the ozone gas may be discharged to the outside through the purification exhaust port.

In one embodiment, the plasma generation device may further include a plasma cap provided at a front end of the plasma tip, a tip of a predetermined material surrounding the plasma cap, and an ampoule storage part which is a predetermined space formed between the plasma tip and the tip.

In one embodiment, from the perspective of a cross-sectional view, a front end of the tip may be formed to be longer outward than the front end of the plasma cap.

In one embodiment, the plasma generation device includes a processor that controls an operation thereof, wherein the processor may control a rotation speed of the driving fan depending on an amount of ozone gas inhaled into the intake port of the ozone intake passage and the presence or absence of ozone contained in the gas discharged through the purification exhaust port.

In one embodiment, the plasma generation device may further include a first sensor that senses the amount of ozone inhaled into the intake port and a second sensor that senses whether ozone discharged through the purification exhaust port is present.

### Advantageous Effects

According to the present disclosure, it may be seen that ozone gas generated while using a plasma generation device is u-turned at the outside, inhaled again through an intake port of the plasma generation device, passed through a filter to remove ozone, and then discharged to the outside. Therefore, it is possible to prevent the ozone from being in direct contact with the skin as much as possible, and furthermore, the ozone-removed gas is discharged, thereby preventing damage caused by the ozone gas.

### BRIEF DESCRIPTION OF THE DRAWINGS

FIGS. 1A and 1B are perspective views showing a plasma generation device according to an embodiment of the present disclosure.

FIG. 2 is a cross-sectional view of the plasma generation device of the present disclosure taken along line I-I′.

FIG. 3 is a cross-sectional view of the plasma generation device of the present disclosure taken along line II-II′.

FIG. 4 is an exemplary view showing a plasma discharge direction and an ozone inhalation direction during use of the plasma generation device of the present disclosure.

FIG. 5 is a block diagram showing a driving system of the plasma generation device of the present disclosure.

### DETAILED DESCRIPTION OF THE EMBODIMENTS

While the present disclosure can be modified in various ways and take on various alternative forms, specific embodiments thereof are shown in the drawings and described in detail. There is no intent to limit the disclosure to the particular forms disclosed. On the contrary, the disclosure is to cover all modifications, equivalents, and alternatives falling within the spirit and scope of the appended claims. When it is deemed that detailed descriptions of related well-known technology might obscure the gist of the present disclosure, those detailed descriptions will be omitted.

Although the terms first, second, etc. may be used to describe various components, these components should not be limited by these terms. These terms are used only to distinguish one component from another.

The terminology used herein is merely used to describe specific embodiments and is not intended to limit the present disclosure. The singular expression includes the plural expression unless the context clearly indicates otherwise. In this application, it should be understood that the terms “comprise” or “include”, when used herein, specify the presence of stated features, integers, steps, operations, elements, and/or components, but do not preclude the presence or addition of one or more other features, integers, steps, operations, elements, components, and/or groups thereof.

Spatially relative terms such as below, beneath, lower, above, and upper may be used to easily describe the relationship between one element or components and another element or components, as depicted in the drawings. The spatially relative terms should be understood as terms including different orientations of the elements during use or operation in addition to the orientation depicted in the drawings. For example, when an element depicted in the drawings is flipped over, an element described as being below or beneath another element may be placed above or upper the other element. Therefore, the exemplary term below may include both the lower and upper directions. Elements may also be oriented in other directions, and thus the spatially relative terms may be interpreted based on their orientation.

Expressions indicating a part, such as “part” or “portion” used in the present disclosure, mean that the corresponding component may indicate a device that can include a specific function, software that can include a specific function, or a combination of a device and software that can include a specific function, but are not necessarily limited to the expressed functions, and various modifications and variations may be made from these base descriptions by those skilled in the art to help a more general understanding of the present disclosure.

Therefore, the spirit of the present disclosure is defined not by the described embodiments but by the appended claims, and encompasses all modifications and equivalents that fall within the scope of the appended claims.

Although a plasma generation device according to an embodiment of the present disclosure may be particularly suitable for use in a portable skin care device and a personal skin care device, the embodiment of the present disclosure is not necessarily limited to such applications.

The plasma generation device according to an embodiment of the present disclosure may be configured such that one side based on a plasma generation pad generating plasma becomes a plasma movement passage, and the other side includes an ozone intake passage in which ozone gas is u-turned to be inhaled again.

According to this embodiment of the present disclosure, plasma generated by the plasma generation pad is transmitted to the skin by flow of air flowing in from the outside, and ozone gas generated when plasma is generated is u-turned from a skin direction to the plasma generation device, inhaled through the ozone intake passage, and discharged to the outside in a state in which ozone is removed while passing through a predetermined filter.

Hereinafter, the present disclosure will be described in more detail based on embodiments illustrated in the drawings.

The plasma generation device according to the present disclosure may be designed in various ways, such as a gun type or a bar type. The main configurations claimed by the present disclosure are designed identically for the gun type and the bar type, and accordingly, in the present embodiment, only a plasma generation device formed in the gun type is described as an example.

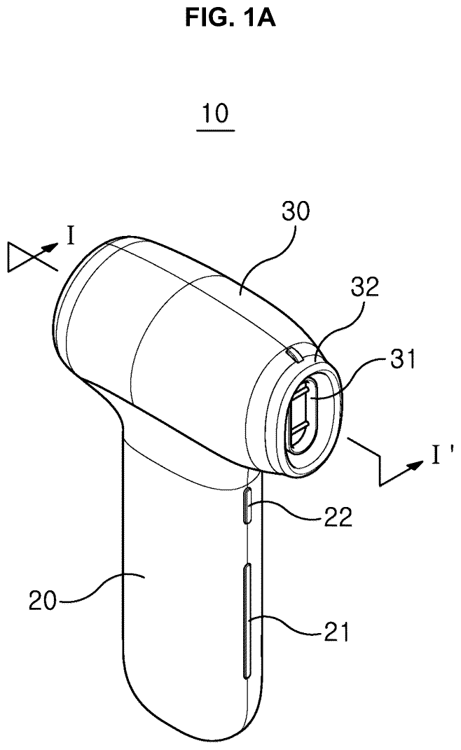

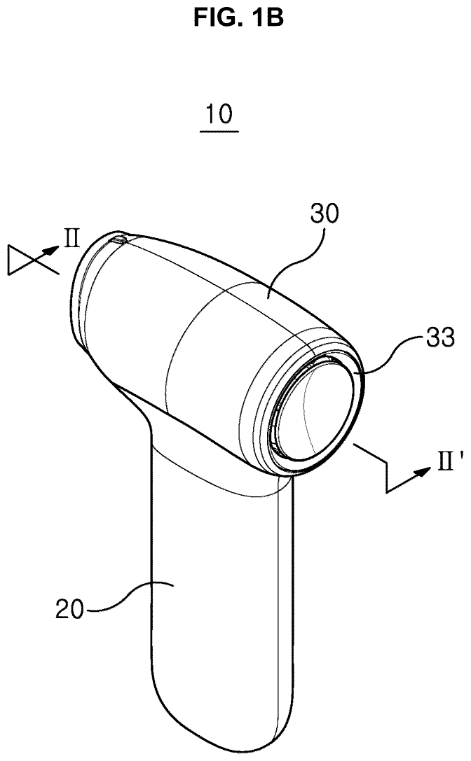

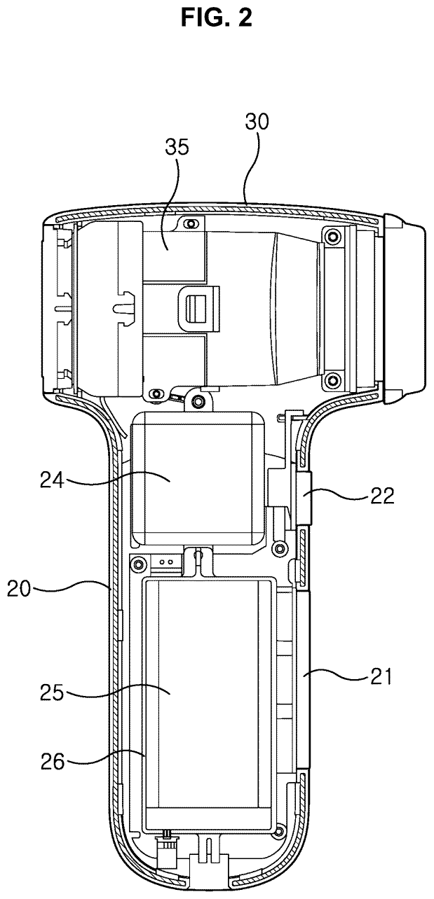

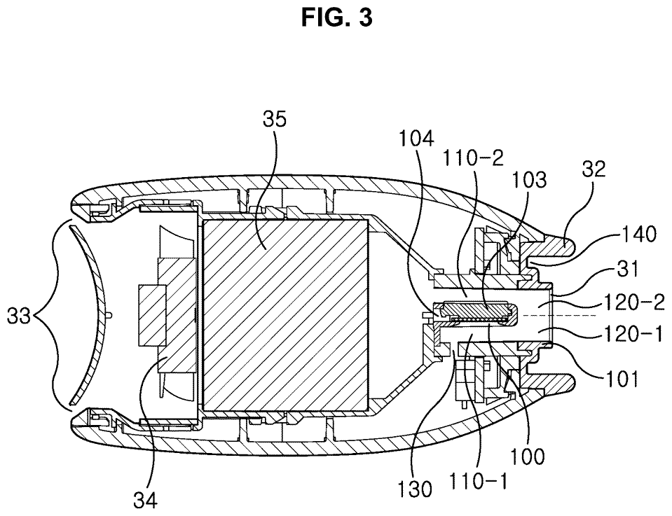

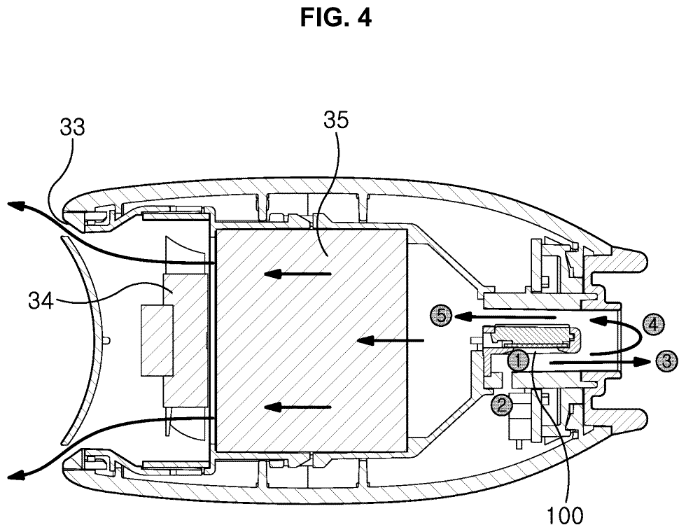

FIGS. 1A and 1B are perspective views showing a plasma generation device according to an embodiment of the present disclosure. FIG. 2 is a cross-sectional view of the plasma generation device of the present disclosure taken along line I-I′. FIG. 3 is a cross-sectional view of the plasma generation device of the present disclosure taken along line II-II′. FIG. 4 is a partial cross-sectional view of the plasma generation device of the present disclosure.

Referring to FIGS. 1A and 1B, a plasma generation device 10 may include a handle 20 and a body 30. The handle 20 and body 30 may be formed integrally.

The handle 20 may be formed in a shape that is easy to grip for a user to use. An LED indicator 21 for indicating battery status and a power button 22 for operation and function may be formed outside the handle 20, and a high-voltage circuit 24, a battery 25, and a PCB circuit board 26 may be provided inside the handle 20 (FIG. 2).

The LED indicator 21 may display different colors depending on the remaining battery capacity. For example, white may be displayed for 90 to 100%, orange for 60 to 90%, and red for 40 to 60%.

A power-on operation is performed by pressing the power button 22 for a predetermined period of time, and a power-off operation is performed by pressing the power button 22 again for a predetermined period of time while a mode is selected. A mode may be selected for each operation of pressing the power button 22 once in a power-on state. The mode includes spot mode, line mode, and face mode.

The body 30 may be provided with a plasma cap 31 and a tip 32 at a front end thereof, and a purification exhaust port 33 may be formed at a rear end of the body. The plasma cap 31 is configured to prevent cosmetics applied to the skin from entering the body when the device 10 of the present disclosure is used in close contact with the skin, and the tip 32 is configured to soften friction when in close contact with the skin. The plasma cap 31 and the silicone tip 32 may be consumables that may be replaced after a certain period of use. Here, the tip 32 may be made of various materials such as silicone, thermoplastic elastomer (TPE), polypropylene (PP), or the like, and above all, any material that is harmless to the human body and soft is acceptable. In addition, the purification exhaust port 33 may be an exhaust port through which gas from which ozone has been purified or removed is discharged.

Referring to FIG. 2, it can be seen that the aforementioned high-voltage circuit 24, battery 25, and PCB circuit board 26 is built in the handle 20 of the plasma generation device 10, and the handle 20 is provided with a user-operable power button 22 and LED indicator 21 on an outer surface thereof. In addition, the plasma cap 31 and the tip 32 are mounted at the front of the body 30, and a driving fan 34 is installed at the rear end 30 of the body to inhale ozone and discharge the ozone-free gas rearward through a purification exhaust port 33.

The plasma cap 31 may be formed, for example in a mesh shape. Plasma may be discharged toward the skin through the plasma cap 31, and the ozone gas generated by the plasma may be inhaled inside the body 30.

As shown in FIG. 3, the plasma generation device 10 includes a plasma generation pad 100 at the front of the body 30. The plasma generation pad 100 is configured to generate plasma and maybe installed inside the plasma tip 101 while being supported by a support bracket 103. At this time, the support bracket 103 is installed long in a lateral direction of the body, and the plasma generation pad 100 is mounted on one side of the support bracket 103. Therefore, the plasma generation pad 100 is configured to generate plasma toward a side surface of the plasma tip 101 rather than toward the front of the body 30 where the plasma cap 31 is located.

The plasma tip 101 and the support bracket 103 may be fixedly installed by a plate 104 provided inside the body 30.

The plasma generation pad 100 may be formed in any size and shape that may be installed on the support bracket 103.

Meanwhile, with the support bracket 103 interposed therebetween, one side serves as a plasma movement passage 110-1 through which plasma moves, and the other side serves as an ozone intake passage 110-2. Based on the drawing, a lower side of the support bracket 103 serves as the plasma movement passage 110-1, while an upper side serves as the ozone intake passage 110-2. In addition, a discharge port 120-1 for discharging plasma is formed in conjunction with the plasma movement passage 110-1, and an intake port 120-2 for drawing in ozone gas is formed in conjunction with the ozone intake passage 110-2.

As such, in the present disclosure, a direction in which plasma is moved and discharged and a direction in which ozone is inhaled and moved are perfectly separated from each other, thereby enabling plasma to be discharged and ozone gas to be inhaled through different passages. In addition, when separating the discharge port 120-1 and the intake port 120-2 in this way, there is an advantage that it is possible to indirectly supply plasma while simultaneously efficiently inhaling ozone gas generated from the plasma.

As shown in FIG. 3, an air inflow passage 130 is formed inside the body 30. The air inflow passage 130 may be a passage that allows external air to flow in front of the plasma generation pad 100. That is, the air inflow passage 130 and the plasma movement passage 110-1 are connected to each other. Plasma generated in the plasma generation pad 100 by external air flowing in front of the plasma generation pad 100 through the air inflow passage 130 may be discharged to the outside through the discharge port 120-1.

According to the present embodiment, the plasma generation pad 100 is installed toward the side surface rather than the front, and the plasma generated by the plasma generation pad 100 is transmitted to the human skin or the like in a flow direction of the external air due to the inflow of external air. Therefore, high-energy charged particles generated from the plasma, which may cause pain and damage to the skin, are prevented from being directly transmitted to the skin or the like. In this way, the plasma may minimize the direct transmission of ozone gas generated as a byproduct of the plasma to the skin while in sufficient contact with the skin or the like. Simultaneously, ozone gas may be removed by the ozone removal function. Accordingly, it can be seen that the damage caused by ozone gas may be minimized.

In FIG. 3, the plasma generation device 10 includes configurations for removing ozone. Specifically, the configurations for removing ozone include a filter 35 and a driving fan 34.

The filter 35 may be provided in a center of the inside of the body 30 behind the plasma generation pad 100. At least one filter 35 may be installed, and when two or more filters 35 are installed, they may be installed continuously between the plate 104 and the driving fan 34.

The filter 35 may be a porous carbon filter, a HEPA filter, or a porous filter, but it is not necessarily limited thereto. Any filter capable of effectively removing ozone gas and other impurities may be used. For example, although not specifically shown in the drawing, the filter may be a filter in which a first HEPA filter, at least one porous filter, and a second HEPA filter are continuously manufactured from the plate 104 toward the driving fan 34. The first HEPA filter may remove impurities included in ozone gas that is generated from the plasma generation pad 100 and inhaled through the ozone intake passage 110-2, the porous filter may remove ozone gas while reducing a rate of movement of ozone gas, and the second HEPA filter may remove impurities generated from the porous filter. In particular, the porous filter may be provided in a structure in which multiple holes are perforated in an ozone filter made of activated carbon (carbon-based) and/or manganese dioxide MnO2 catalyst materials which are materials capable of removing ozone.

The plasma generation device 10 according to the present embodiment is a device that improves the skin by removing bacteria or the like by treating air plasma on the skin surface. When using the plasma generation device 10, the user uses it in close contact with the skin or the like, and thus, when the user uses gel-type or mist-type cosmetics, there is a possibility that the cosmetic ingredients (hereinafter referred to as “ampoules”) may block the discharge port 120-1 through which plasma is discharged or the intake port 110-2 through which ozone is inhaled. In this case, normal use of the plasma generation device 10 may be impossible.

The present embodiment provides an ampoule storage space 140 in order to prevent this. The ampoule storage space 140 may refer to a space designed to store ampoules, i.e., cosmetics, etc., separately without blocking the discharge port 120-1 or the intake port 120-2. Specifically, the ampoule storage space 140 may be a space between the plasma tip 101 and the tip 32.

When using the plasma generation device 10, ampoules may be stored in the ampoule storage space 140 because one end of the tip 32 is formed to be longer than that of the plasma tip 101. That is, when the plasma generation device 10 is used while in close contact with the skin, an end of the tip 32 scratches the ampoule first and may be stored directly in the ampoule storage space 140.

In the present embodiment, a protrusion (not shown) may be further formed at a front end of the plasma tip 101 to prevent the ampoule stored in the ampoule storage space 140 from flowing toward an inlet side.

FIG. 4 is an exemplary view showing a plasma discharge direction and an ozone inhalation direction during use of the plasma generation device of the present disclosure.

Referring to FIG. 4, the user operates the power button 22 to turn on the plasma generation device 10, and while in close contact with the skin, the user uses the plasma generation device 10 to perform a skin massage or the like.

Then, the plasma generation pad 100 operates by a driving voltage applied from the high-voltage circuit 24 to generate plasma \textcircled{1}, and simultaneously, external air flows in through the air inflow passage 130 \textcircled{2}. The plasma radiated in front of the plasma generation pad 100 by flow of the external air flowing in through the air inflow passage 130 is discharged toward the skin through the discharge port 120-1 \textcircled{3}. That is, the plasma flows to the discharge port 120-1 while changing its flow direction in a perpendicular direction due to the flow of air flowing in from the outside. Furthermore, the plasma transmitted to the skin through the discharge port 120-1 may remove bacteria or the like.

Meanwhile, when plasma is generated, ozone gas is generated as a byproduct thereof. The generated ozone gas should be removed. The ozone gas is u-turned by driving of the driving fan 34 and is inhaled through the intake port 120-2 \textcircled{4}, and then may be inhaled into the ozone intake passage 110-2. Then, ozone inhaled through the ozone intake passage 110-2 is supplied to the filter 35 located at the rear end \textcircled{5}, and the filter 35 decomposes and removes ozone. In the present embodiment, the filter 35 may decompose ozone gas to less than 0.050 PPM.

When ozone gas passes through the filter 35, ozone is removed, and the ozone-removed gas is discharged to the outside through the purification exhaust port 33.

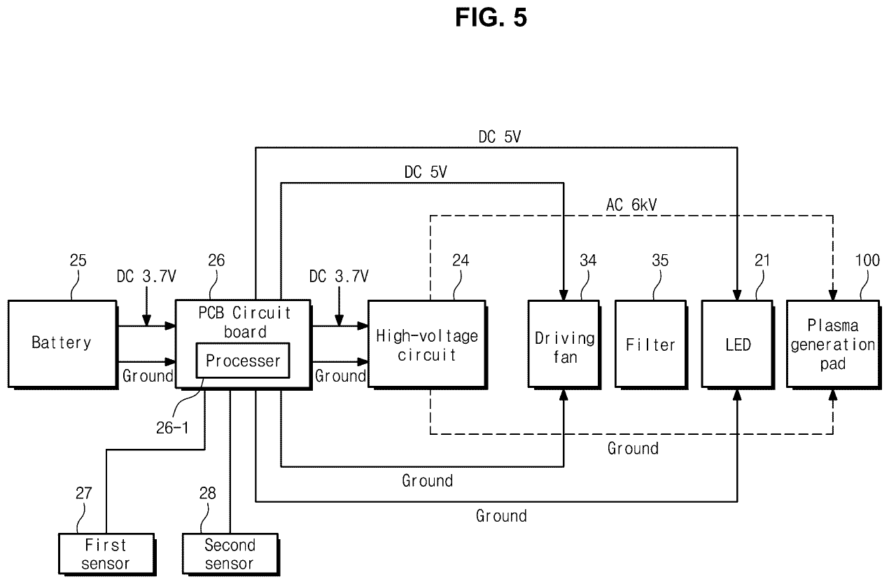

FIG. 5 is a block diagram showing a driving system of the plasma generation device of the present disclosure.

Referring to FIG. 5, the driving system may include a battery 25, a PCB circuit board 26, a high-voltage circuit 24, a driving fan 34, a filter 35, an LED 21, a plasma generation pad 100, and the like.

The battery 25 may deliver a driving voltage, for example, DC 3.7 V, to the PCB circuit board 26.

The PCB circuit board 26 may convert and supply a voltage supplied by the battery 25 into a respective driving voltage for driving the high-voltage circuit 24, the driving fan 34, and the LED 21. The high-voltage circuit 24, the driving fan 34, and the LED 21 may be driven by mutually different driving voltages, respectively, and may be 3.7V, 5V, and 5V, respectively in the embodiment. That is, the PCB circuit board 26 may supply DC 3.7 V for driving the high-voltage circuit 24 and may supply DC 5 V for driving the driving fan 34 and the LED 21.

In the present embodiment, the PCB circuit board 26 may include a processor 26-1. The processor 26-1 may also perform a function of controlling a speed of the driving fan 34 in addition to a function of converting the driving voltage. The speed of the driving fan 34 varies depending on the user's button operation, and the processor 26-1 adjusts the speed of the driving fan 34 according to the button operation. Here, the processor 26-1 may be implemented as a digital signal processor (DSP) that processes digital signals, a microprocessor, or a time controller (TCON). However, the present disclosure is not limited thereto, and the processor may include at least one of a central processing unit (CPU), a microcontroller unit (MCU), a micro processing unit (MPU), a controller, an application processor (AP), a graphics-processing unit (GPU), a communication processor (CP), and an address resolution protocol processor (ARP), or may be defined by the relevant term. Furthermore, the processor 26-1 may be implemented as a system on chip (SoC) with a built-in processing algorithm and a large-scale integration (LSI), or may be implemented in a form of a field programmable gate array (FPGA).

The plasma generation pad 100 is driven by a voltage supplied by the high-voltage circuit 24.

Meanwhile, the plasma generation device 10 of the present disclosure may further add an ozone amount detection sensor 27 (first sensor) to the intake port 120-2 side. Furthermore, the processor 26-1 may adjust a rotation speed of the driving fan 34 depending on a sensing value of the ozone detection censor 27 to control an ozone gas intake amount. In this case, when the detected intake amount increases, the driving fan 34 may be rotated more rapidly.

As another example, an ozone detection sensor 28 (second sensor) may be added to the purification exhaust port 33. The ozone detection sensor 28 senses the presence of ozone in the gas discharged through the purification exhaust port 33, and the processor 26-1 may adjust the rotation speed of the driving fan 34 depending on the sensed value. Therefore, when ozone is detected, a time when ozone gas stays in the filter 35 is increased by reducing the rotation speed of the driving fan 34, thereby removing more ozone.

As described above, in the present disclosure, it can be seen that while indirectly supplying plasma to the skin, ozone gas generated by the plasma is u-turned at the outside and quickly inhaled into the device again to remove it.

While the present disclosure has been described with reference to the illustrated embodiments, these are merely exemplary, and it will be understood by those skilled in the art that various alterations, variations, and equivalent other embodiments may be made without departing from the gist and scope of the present disclosure. Therefore, the scope of the present disclosure should be defined by the technical spirit of the appended claims.

What is claimed is:

1. A plasma generation device comprising:
a handle and a body,
wherein the body includes:
a plasma tip formed at a front end of the body; and
a plasma generation pad located inside the plasma tip and generating plasma toward an inner surface of the plasma tip,
wherein one side based on the plasma generation pad is used as a plasma movement passage for moving the plasma so that the generated plasma is transmitted to the outside through a discharge port, and the other side is used as an ozone intake passage through which ozone gas additionally generated by the plasma is u-turned at the outside to be inhaled through an intake port.

2. The plasma generation device of claim 1, wherein the plasma generated by the plasma generation pad is discharged to the outside through the discharge port by vertically changing a direction in the plasma movement passage.

3. The plasma generation device of claim 1, wherein the body further includes an air inflow passage through which external air flows in, and
the air inflow passage is connected to the plasma movement passage.

4. The plasma generation device of claim 1,
wherein the body further includes:
a purification exhaust port formed at a rear end of the body; and
a filter and a driving fan sequentially disposed between a plate and the purification exhaust port, and
ozone is removed from the ozone gas inhaled into the ozone intake passage by a rotational force of the driving fan through the filter and the ozone gas is discharged to the outside through the purification exhaust port.

5. The plasma generation device of claim 1, further comprising:
a plasma cap provided at a front end of the plasma tip;
a tip surrounding the plasma cap; and
an ampoule storage part which is a predetermined space formed between the plasma tip and the tip.

6. The plasma generation device of claim 5, wherein, from the perspective of a cross-sectional view, a front end of the tip is formed to be longer outward than the front end of the plasma cap.

7. The plasma generation device of claim 4, comprising
a processor that controls an operation of the plasma generation device,
wherein the processor controls a rotation speed of the driving fan depending on an amount of ozone gas inhaled into the intake port of the ozone intake passage and the presence or absence of ozone contained in the gas discharged through the purification exhaust port.

8. The plasma generation device of claim 7, further comprising:
a first sensor that senses the amount of ozone inhaled into the intake port; and
a second sensor that senses whether ozone discharged through the purification exhaust port is present.
